(19) United States
(12) Patent Application Publication
(10) Pub. No.: US 2005/0191677 A1
(43) Pub. Date: Sep. 1, 2005

(54) Mass spectrometric concentration measurement of proteins

(75) Inventors: Jochen Franzen, Bremen (DE); Detlev Suckau, Grasberg (DE)

Correspondence Address: KUDIRKA & JOBSE, LLP, ONE STATE STREET, SUITE 800, BOSTON, MA, 02109, US

(73) Assignee: BRUKER DALTONIK GMBH, Bremen (DE)

(21) Appl. No.: 11/054,621

(22) Filed: Feb. 9, 2005

(30) Foreign Application Priority Data
Feb. 12, 2004 (DE) 10 2004 007 005.9

(51) Int. Cl. C12Q 1/68; G01N 33/553

(52) U.S. Cl. 435/6; 436/526

(57) ABSTRACT

The invention relates to the determination of the relative concentrations of proteins or protein derivatives in liquids. The invention provides a method which uses nanoparticles coated with specific affinity collectors in order to fish the desired proteins or protein derivatives out of the liquids and to separate them, in order to introduce them to the mass spectrometric frequency analysis after elution from the affinity collectors. This makes it possible to determine the concentrations of several proteins or several forms of protein modification or mutation relative to each other with relatively high measuring dynamics.

### FIELD OF THE INVENTION

The invention relates to the determination of the relative concentrations of proteins or protein derivatives in liquids.

### BACKGROUND OF THE INVENTION

In modern proteomics, the focus of interest has shifted more and more towards the determination of the concentrations of various proteins or peptides (small proteins) relative to each other or the determination of the relative concentrations of different derivatives of the same protein. With different derivatives of the same protein the reference is not only to different posttranslational modifications such as phosphorylation or glycosylation but also to forms changed by mutation which frequently occur in the same individual in both allele forms inherited from father and mother, and often with different frequencies. In addition, there are different forms of the same protein generated by splice variation and also larger breakdown products (proteolytic fragments) of a protein. The various breakdown forms of a protein and their relative frequency are of particular interest when one is concerned with products with several competing breakdown paths, where one of the breakdown paths provides toxic, diagnostically relevant or other pathogenic forms. A known example is the abnormal breakdown of a type of protein molecule called a “prion” in the brains of cows, which leads to the crystallizing of the breakdown products and hence to “mad cow disease” or BSE. A similar phenomena is observed with Alzheimer's disease.

The type of frequency determination most often used until now has been the “expression analysis” which serves to determine the relative concentrations of proteins in “sick” or “stressed” samples relative to “healthy” samples. It is usually based on the two-dimensional separation of proteins by 2D gel electrophoresis followed by staining of the proteins. The determination of the relative concentrations here is carried out either photometrically via the intensity of the staining or via mass spectrometric measurements, the latter also permitting an identification of the proteins. The indisputable and particular advantage of 2D gel electrophoresis lies in finding over and under expressions of proteins for which such reactions had been previously unknown, but the limited concentration range means that this type of expression analysis is becoming ever less important.

Both the expression analysis using 2D electrophoresis and the chromatographic methods can only measure the frequently occurring proteins; the measurement here relates roughly to the upper three to four powers of ten of the concentration range of proteins (10⁷ to a few 10¹⁰ picograms per milliliter), whereas the complete analytically interesting concentration range is estimated to be more than ten powers of ten. In blood plasma, there are important control proteins and signal messengers which are of significant interest only in the lower concentration ranges; the important interleukins, for example, are to be found in concentrations of around one to three picograms per milliliter at the lower limit of the analytically interesting range. For molecular weights between 10⁴ and 10⁵ grams per mol, the concentrations of the interleukins are around 10 to 100 attomol per milliliter. The breakdown products of cell proteins emerging from cells, which are of great interest for diagnostic purposes, occur in the plasma in concentrations of around 10² to 10⁴ picogram per milliliter (N. L Anderson and N. G. Anderson, “The human plasma proteome”, Mol. Cell Proteomics 1, 845-867 (2002)).

The removal of highly concentrated proteins such as albumins and globulins in order to be better able to measure the proteins which are not as highly concentrated, is generally considered to be extremely questionable since many proteins present in low concentrations tack themselves onto the highly concentrated proteins in the form of non-covalently bonded complexes and are removed with these.

Nor do two-dimensional chromatographic or electrophoretic separation methods help to significantly enlarge the dynamic measuring range in the absence of special biochemical measures. Methods which bond specific affinity tags such as biotin derivative to selected proteins in order to be able to use immobilized affinity collectors to collect them out of larger volumes of liquid represent one possibility. However, here as well, it is not possible to select proteins individually but only on the basis of specific chemical groups which, in turn, are inevitably present in more or less all proteins.

A further method for fishing specific, predetermined proteins has therefore developed: the capture (or “fishing”) of proteins using antibodies firmly bonded (immobilized) to surfaces. Antibodies very specifically bond only certain proteins although here, as well, cross-reactions with other proteins occasionally occur. In principle, this method has been known for a long time for the mass spectrometric analysis of individual proteins, but because production of the antibodies was previously protracted and expensive, it was not used very often. An early example of this is the work of Detlev Suckau et al., “Molecular epitope identification by limited proteolysis of an immobilized antigen-antibody complex and mass spectrometric peptide mapping”, Proc. Natl. Acad. Sci. USA, 87 (1990) 9848-9852.

Of late, it has also been possible to achieve the effect of antibodies which have molecular weights from 150 000 to 190 000 atomic mass units, using computer-modeled peptides in the range of only 20 amino acids (only about 2400 atomic mass units) and therefore to be able to substitute the expensive antibodies with inexpensive peptides which can be synthesized. There is a risk of increased cross-reactivity, however. Other specifically effective interaction partners are also known, such as lectins, metal chelates for phosphate binding (IMAC), protein nucleic acids (PNAs), oligonucleotides, inhibitors, receptors, ligands and others.

Recently, so-called chip arrays have frequently been used for the capture of individual proteins, these chip arrays are coated in individual fields from 0.01 to 1 mm² in size with various types of antibodies or other affinity collectors. This makes it possible to fish for whole families of proteins such as the kinases, for example, and in favorable cases to determine their abundances relative to each other. The relationships between individual kinases can be characteristic for certain diseases, in which case they are termed “biomarkers”. The kinases are to be found in a low concentration range.

We are dealing here with the extensive field of so-called chip arrays with covalently bonded capture substance molecules to detect affinity binding biopolymer molecules. The capture substance molecules bonded on the array fields can be DNA molecules (“DNA chips”), protein molecules (“protein chips”) or other types of molecules with a specific affinitive binding capability. In the following, the proteins sought will be designated as “analyte molecules”, and the capture substance molecules on the chip fields simply as “capture molecules” or also as “probe molecules”. The specific affinitive binding of the analyte molecules to the probe molecules takes place out of solutions in which the analyte molecules sought could occur, the requirement being that the solutions must be in direct contact with the surface of the coated chip.

These chip arrays with probe molecules are used quite generally for the study of the bonds (for example cross-reactions in antibody bonding), but in particular for the selective capture of analyte molecules from body fluids and hence for the qualitative and, to a limited extent, quantitative analysis of these analyte molecules. In a few cases, for example for the detection of identifying DNA strands of pathogens, the analyses are limited to simple statements concerning the presence or absence of the pathogen. As a result of the large number of probe fields on the chip arrays, the presence of one or more pathogens among many different types of pathogen can be detected simultaneously in a body fluid sample.

The analysis methods with such chip arrays are termed “cell-based assays”; the methods themselves are frequently termed “screening”. The chip arrays have significant disadvantages, however. Since the fields on the arrays are very small, they can only bond limited amounts of analyte molecules. If the fishing coats them to saturation, it is still possible to carry out a qualitative analysis of the type of protein captured, but a quantitative analysis, i.e. the determination of relative concentrations, is lost. Since, on the other hand, however, only few bonded analyte molecules on a chip field can be detected only with the greatest of difficulty, the dynamic range of the measurement of this method of fishing with chip arrays is very small; depending on the detection method used it amounts to only one to three powers of ten.

One advantage of the chip array is that it is not limited to mass spectrometric detection alone. To date, several methods have established themselves as the prior art for the detection of the bonding of analyte molecules to probe molecules but they will only be explained briefly here.

One way of detecting the bonds is by additional fluorescent dyes bonded to the analyte molecules, for example; the fluorescent dyes suitable for this are expensive, however. It is also possible to bond the fluorescent dyes to the capture molecules; the bonds are then detected by measuring the “quenching” or the measurement of a slight frequency shift of the dye on being captured by analyte molecules.

A further method, which is being developed at present, consists of the simultaneous bonding of the analyte molecules and larger masses, for example by nanoparticles, to suitable oscillators to detect the affinitive binding by means of surface acoustic waves (SAW), whose frequency is a function of the coating.

The method of plasmon resonance spectrometry, also used as a means of detecting the affinity binding of analyte molecules to probe molecules, requires somewhat larger areas for the flat reflection of the light in each case, so that it has not yet been possible to produce arrays with larger numbers of fields for this type of detection. The advantages lie in the fact that it is also possible to measure the kinetics of the bonding process.

The detection methods named have the advantage of somewhat larger measuring dynamics, amounting to differences in concentrations of around three to four powers of ten, yet they also have the disadvantage that an independent identification of the proteins captured does not occur. Since all antibody bonds also involve cross-reactions with other proteins, one can never be sure of having captured the correct protein or a particular derivative. This type of independent identification is reserved solely for mass spectrometry. The use of mass spectrometry is even more imperative when different forms of a protein, which are all captured by the same monoclonal antibody, shall be measured as a ratio. The use of polyclonal antibodies, which is also of interest for analytical purposes, also compels one to use the mass spectrometric means of detection.

Although mass spectrometric detection of the affinity binding of analyte molecules, for example with ionization by means of matrix-assisted laser desorption and ionization (MALDI) after the addition of appropriate matrix substances, is also very expensive because of the mass spectrometer required, it does have the advantage of providing an additional confirmation of the identity of the analyte molecules by means of their exact mass. This invaluable advantage conflicts with the disadvantage of low measuring dynamics, which amount to only around two powers of ten. On a large array field with an area of one square millimeter, with dense, monomolecular coating, only one picomol of analyte molecules can be captured, in general, however, only a tenth of this number is possible, i.e. around 100 femtomol since, to allow sufficient steric freedom for the capture, the coating must be much less than one monomolecular layer. The mass spectrometric detection limit for MALDI ionization which can be achieved in practice is around one femtomol, however, resulting in a measurement range of around two powers of ten.

Furthermore, it is difficult to fish out proteins occurring in low concentrations from larger volumes of liquid using chip arrays. For the interleukins, for example, around 10 to 100 milliliters of blood plasma must be fished for one femtomol of interleukin, a task which chip arrays have not yet been able to perform.

A method is therefore required which provides, on the one hand, an independent identification and, on the other, a broad measuring range for the concentration determinations.

### SUMMARY OF THE INVENTION

The invention provides a method which uses nanoparticles coated with affinity collectors in order to fish the desired proteins or protein derivatives out of the liquids and to separate them, in order to introduce them to a mass spectrometric frequency analysis after elution from the affinity collectors. This makes it possible to determine the concentrations of several proteins or several forms of protein modification or mutation relative to each other with relatively high measuring dynamics. The invention includes adding nanoparticles coated with capture molecules for the proteins or protein derivatives to be investigated to a sample solution, thereby binding the proteins or protein derivatives affinitively to the capture molecules. The nanoparticles are then separated from the sample solution, and the the proteins or protein derivatives are eluated from the nanoparticles. Thereafter, the eluate is submitted to the mass spectrometric measurement and the required concentration ratios of the proteins or protein derivatives are determined from the results of the mass spectrometric measurement.

In practice, the nanoparticles of the solution may added as a suspension. Antibodies or synthetic specifically bonding molecules, lectins, metal chelates, protein nucleic acids, oligonucleotides, inhibitors, receptors or ligands, for example, may be used as capture molecules on the nanoparticles. Separation of the nanoparticles from the solution may be by filtration, by centrifuging, sedimentation or, in the case of magnetizable nanoparticles, by the application of a magnetic field. A washing step for the separated nanoparticles may also be included.

In accordance with the invention, a mixture of nanoparticles, each coated with capture molecules for one of the proteins, may be used to determine the concentration ratio of several proteins. The mixture may include non-magnetizable and magnetizable nanoparticles coated each with capture molecules for a specific protein, which may be separated out of the solution independently, and both types of nanoparticles or their eluates may be mixed in a ratio such that the resulting concentration ratio of the two proteins lies within the dynamic measuring range of the mass spectrometer. One or more internal calibrants for one or more proteins or derivatives may also be added, with the calibrants of the proteins or derivatives exhibiting distinguishable masses, and the addition being able to be carried out before the binding to the nanoparticles or after the elution. The eluate may also be subjected to a chromatographic or electrophoretic separation of its constituents before the mass spectrometric measurement.

The central idea of the invention is not to use a chip array for the capture of different analyte molecules when measuring the concentration ratios of different proteins or different protein derivatives of a protein, but rather to use nanoparticles coated with capture molecules, preferably in the form of small spheres in the range of 500 to 1500 nanometers in diameter, more generally as particles of any shape from 10 to 10000 nanometers in size and, after elution, to introduce the analyte molecules captured to a mass spectrometric measurement to determine the concentration ratios. With this type of capture, one loses the field or cell-based differentiation as is present in chip arrays and as is required for a substance-blind bond detection using fluorescence, plasmon resonance or SAW. Mass spectrometry can, however, use the different masses to separately detect different types of proteins or protein derivatives which are present in the same sample after elution from the capture molecules, and thus even ascertain their identity with a high degree of reliability.

The nanoparticles preferably have diameters of slightly less than a micrometer; these can then form suspensions in liquids which remain suspended for a long period. One milligram of the nanoparticles has a surface area of tens of square centimeters, i.e. an area which is easily more than a thousand times greater than the surface of any field of a chip array; it can easily be selected to be larger than required. Moreover, an invaluable advantage of the nanoparticles is that their amount can be adapted to suit the analytical problem by pure pipetting of the suspension. A method such as this is termed a “scaleable” method. The concentrations of the suspensions can be adapted for adding to smaller and larger sample volumes. Turbulent stirring or tilting produces an extremely good contact and a relatively rapid capture of the analyte molecules. Magnetizable nanoparticles with a diameter of 900 nanometers, for example, can then be held at the wall of the sample vessel by means of an inhomogeneous magnetic field in order to exchange the sample liquid for a washing liquid and, after sufficient washes, for the addition of a small amount of elution liquid. This is added to the mass spectrometric analysis. The particles can also easily be filtered out or centrifuged to sediment them, in which case non-magnetizable particles can also be used.

The method is automatically linked to a concentration of the desired proteins, which can amount to many powers of ten. If, for example, the sample volume is 100 milliliters, and if the nanoparticles are eluted with only ten microliters, then the concentration increases by four powers of ten.

To capture different types of proteins, a mixture of nanoparticles with different types of coatings, each specific to one type of protein which is to be captured, can be used; nanoparticles with mixed coatings can also be used. The mixtures can easily be adapted to the analytical problem.

To capture different protein derivatives of the same protein, it is possible to use either monoclonal antibodies or polyclonal antibodies. Mixtures of specific antibodies for different types of the protein can also be used. If the protein derivatives are captured by the same antibody, mass spectrometry is the only system of detection which can be used. Chip arrays are of no use whatsoever here. Instead of the antibody, other specific affinitively binding molecules can also be used, for example peptides with a particular design, which can be calculated with the aid of computers nowadays, or other specifically effective interaction partners, such as lectins, metal chelates for phosphate binding (IMAC), protein nucleic acids (PNAs), oligonucleotides, inhibitors, receptors, ligands and others.

By using a mixture of non-magnetic and magnetic nanoparticles, each coated with specific affinity capture molecules and which can be separated and then mixed in various ratios independently of the sample liquid, the concentration ratio can be adapted to the dynamic measuring range of the mass spectrometer.

### DETAILED DESCRIPTION

The first description is of a method which particularly emphasizes the advantages of using mass spectrometry: it relates to the ratio determination for different derivatives of a single protein in an organism or a part of an organism, whereby the different derivatives can be fished out of a liquid sample either together with only one type of antibody or with a single other type of affinity capture molecule. The liquid sample can be a body fluid or it can be produced as cell lysate from a tissue. The sample can originate from a human, animal, plant, single cell or virus. The ratio can be characteristic of a particular disease or stressed state of the corresponding living thing; this is then known as a “biomarker”.

As already described above, the different derivatives of the protein can be different posttranslational modifications such as phosporylation or glycosylation, or also various types of genetic mutation which manifest themselves in a change of the amino acid sequence in the chain molecule. Different splice variants are also be referred to here as derivatives. As long as the mutation does not change the binding motif, the so-called “epitope”, the mutated forms, the so-called “mutants”, are captured in the same way as the so-called “wild type”. The same is true for the modifications, which usually do not bring about a change to the binding epitope. It is then the task of the quantifying and, in this case, also qualifying mass spectrometric analysis, which measures different masses for the various forms of modification, mutation or splice variants, to distinguish which modification or mutation is present. (More precisely: in a mass spectrometer, it is always the different ratios of mass to charge which are measured; however, since in this case the most commonly used type of ionization by matrix-assisted laser desorption (MALDI) usually provides only singly-charged ions, the term “mass” will be used on its own below.)

We also speak here of different derivatives of the protein for the purpose of the invention when referring to the first stages of a metabolic breakdown (ubiquitinylation, enzymatic breakdown) of proteins, as long as the binding epitope is still intact. In a number of cases, these first stages of the breakdown are very interesting biomarkers, since misdirected breakdown can lead to dramatically pathogenic products, as has been established in the case of BSE or Alzheimer's disease.

Therefore, in order to measure the concentration ratios of various protein derivatives in a liquid sample, a pre-determined amount of a suspension with nanoparticles is pipetted into the sample, the nanoparticles here being coated with capture molecules. The nanoparticles are preferably magnetizable. Suspensions of magnetizable nanospheres (“magnetic beads”) 900 nanometers in diameter have already proven extremely successful for other applications; suspensions of these beads remain useable for a long time. The capture molecules can be monoclonal antibodies or molecules having a similar specificity, for example. Care must be taken that the nanoparticles are not coated to saturation for any of the protein derivatives to be measured.

The liquid sample is intimately mixed with the suspension and kept slightly in motion in order to bring all dissolved analyte molecules into contact with the capture molecules.

The mini-particles are then separated from the liquid. Magnetic mini-particles can be drawn to the wall of the vessel by a strong permanent magnet, for example. For this purpose, the vessel should not be overly elongated, since the magnetic effect only extends over some five to ten millimeters. In this case also, careful stirring or tilting helps to bring all particles slowly into the effective range of the magnet and hence to finally capture them in clusters on the wall. For vessels with larger volumes, shapes which are more thin in one dimension are also suitable. For even larger volumes, centrifuging or filtration can be used. The liquids can also be guided through a hose over the magnets.

The collections of particles adhering to the wall or sedimented are then released from the sample solution by either pouring them off or pipetting them, and a washing liquid is added. The particles are washed by removing the magnet and stirring. The washing process can be repeated several times, if necessary. Finally, an eluting liquid is added to the particle collection, which is largely free of liquid, this liquid separates the proteins from the antibodies or other types of capture molecules. Eluting liquids of this type are usually strong, polar organic solvents such as acetone, acetonitrile or alcohols. The eluting liquids with the proteins are then introduced to the mass spectrometric measurement.

Suitable mass spectrometers are those with MALDI ion sources and also those with electrospray ion sources (ESI). In the case of MALDI mass spectrometers, the eluate is spiked with a suitable matrix and dried on a sample support. The solid sample on the sample support is then bombarded with flashes of laser light in the ion source of the mass spectrometer; the ions created are detected in an ion detector separated according to their mass and their number is measured. The eluate can be introduced to, and measured by, a mass spectrometer with electrospray ion source (ESI) either directly or separated again using a chromatograph. In the case of ionization by means of MALDI, a chromatographic separation can also be carried out first.

For ionization by matrix-assisted laser desorption (MALDI), the mini-particles can also be applied directly to a sample support plate. There they can be spiked with a matrix solution and then dried. The matrix solution here acts as an eluting liquid, crystals are formed with encapsulated proteins.

In both cases (MALDI and ESI), measurements of the mass and the intensity produce the desired starting values for accurate identification and determination of the ratio. It could be necessary here to calibrate the ratio with calibration solutions with known ratios. The remaining sample liquid can be tested for remaining protein molecules with a fresh (or a recovered) particle suspension. If protein molecules still occur here, this can be an indication of saturation in the first stage of capture. The occurrence of saturation interferes with the determination of the concentration ratios.

If the posttranslational modifications in question are glycosylations, then a linear distribution of the glycogroups can be measured mass spectrometrically. The linear distributions can be extremely characteristic of the state of stress of the organism. It is also possible, however, to split off the glycogroups down to the basic group by means of a glycosidase and thus only measure one ratio of glycosylated to non-glycosylated proteins.

Another embodiment of the method relates to the measurement of the concentration ratios of two or more different proteins, for example several interleukins in plasma, which provide information concerning the state of stress of a body caused by different types of inflammation. Mixtures of particle suspensions containing particles with different types of capture molecule coatings are used for this. The different types of coating can be composed of different types of particles, each coated with one type of capture molecule, or they can contain the same type of particle mixed in a single coating. If one has several particle suspensions coated solely with capture molecules of a single kind, it is then simple to produce any mixture required.

The rest of the procedure is the same as described for the method above: add the suspension, stir, remove the sample liquid after collecting the particles, wash, eluate, mass spectrometric measurement, determination of the ratio or ratios.

The special feature when measuring the concentration ratios of the diagnostically extremely interesting interleukins is the fact that they are present in the plasma in very low concentrations. The interleukins must be fished out of around 100 milliliters of plasma in order to obtain an amount which exceeds the mass spectrometric detection limit. This fishing can only be carried out successfully with the method according to the invention presented here.

The particle suspensions can be reactivated again by washing the particles in eluting liquid. Since antibodies are extremely expensive, recovery is worthwhile.

If concentration ratios are to be measured in the eluate which exceed the dynamic measuring range of the mass spectrometer, a special method can be used in which a mixture of magnetic and non-magnetic nanospheres are used. The two types of nanoparticles have different coatings with capture molecules for different types of analyte molecules. After being fished out, the magnetizable mini-particles can be separated from the non-magnetizable mini-particles by means of a magnetic field, making it possible to alter the mixing ratio of the types of particle, and hence the ratio of the two types of analyte molecule captured, on a broad scale so as to bring the analyte molecules of the two types whose ratio is to be measured to within the measuring range of the mass spectrometer.

An example may serve to explain this: the concentration ratio of two proteins α and β in a blood plasma solution is to be determined, whereby it is to be expected that the protein α in the plasma solution is around 10000 times more concentrated than protein α. One hundred milliliters of the plasma solution are spiked with one milliliter each of a suspension A and a suspension B. Suspension A contains non-magnetic mini-particles with capture molecules for protein α, suspension B contains magnetic beads with capture molecules for protein β. After the affinitive binding of the proteins α and β, the magnetic beads of suspension B are first separated off by a strong magnet, washed and resuspended in a further washing liquid. The remaining solution with the mini-particles of suspension A is now freed from the mini-particles by centrifuging; these mini-particles are then resuspended in 100 milliliters of a washing liquid. Ten microliters are now pipetted out of this liquid with the suspended mini-particles A and added to the washing liquid with the mini-particles B. The mini-particles are now centrifuged out together; the elution of the proteins from this particle mixture should now lead one to expect a ratio of the proteins α and β of only 1:1. A deviation from this can be used to determine the original ratio. The ratio 1:1 can be optimally measured mass spectrometrically, possibly after a calibration.

The proteins from both types of nanoparticle can also be eluted separately, and the eluate liquids then mixed in the desired ratio.

For a mass spectrometric determination of the concentration ratios it is usually necessary, as already explained above, to determine the different types of ionization probabilities using a calibration with known ratios. These techniques, which can also be conducted with isotope-labeled proteins, for example, are known to the specialists in this field, however, and a detailed description is therefore not required.

1. Method for measuring the concentration ratios of various proteins or various protein derivatives of a protein in a sample solution comprising the following steps:
a) add nanoparticles coated with capture molecules for the proteins or protein derivatives to be investigated to the sample solution, thereby binding the proteins or protein derivatives affinitively to the capture molecules,
b) separate the nanoparticles from the sample solution,
c) eluate the proteins or protein derivatives from the nanoparticles,
d) submit the eluate to the mass spectrometric measurement, and
e) determine the required concentration ratios of the proteins or protein derivatives are from the results of the mass spectrometric measurement.

2. Method according to claim 1, wherein the nanoparticles of the solution are added as a suspension.

3. Method according to claim 1, wherein antibodies or synthetic specifically bonding molecules, lectins, metal chelates, protein nucleic acids, oligonucleotides, inhibitors, receptors or ligands are used as capture molecules on the nanoparticles.

4. Method according to claim 1, wherein the nanoparticles in step (b) are separated from the solution by filtration, by centrifuging, sedimentation or, in the case of magnetizable nanoparticles, by the application of a magnetic field.

5. Method according to claim 1, wherein step (b) is followed by one or more steps with washing processes for the separated nanoparticles.

6. Method according to claim 1, wherein a mixture of nanoparticles, each coated with capture molecules for one of the proteins, is used to determine the concentration ratio of several proteins.

7. Method according to claim 1, wherein a mixture of non-magnetizable and magnetizable nanoparticles coated each with capture molecules for a specific protein, is used to determine the concentration ratio of two proteins having a very large concentration ratio, the magnetizable and the non-magnetizable nanoparticles are separated out of the solution independently, and both types of nanoparticles or their eluates are mixed in a ratio such that the resulting concentration ratio of the two proteins lies within the dynamic measuring range of the mass spectrometer.

8. Method according to claim 1, wherein one or more internal calibrants for one or more proteins or derivatives are added, with the calibrants of the proteins or derivatives exhibiting distinguishable masses, and the addition being able to be carried out before the binding to the nanoparticles or after the elution.

9. Method according to claim 1, wherein the eluate is subjected to a chromatographic or electrophoretic separation of its constituents before the mass spectrometric measurement.
